(19) United States
(12) Patent Application Publication
(10) Pub. No.: US 2011/0021940 A1
(43) Pub. Date: Jan. 27, 2011

(54) INCENTIVE AUDIO FOR PULMONARY FUNCTION DIAGNOSTICS

(76) Inventors: Edmond Chu, Erie, CO (US); Rich Rosenthal, Superior, CO (US); Larry Murdock, Alta Loma, CA (US)

Correspondence Address: COOLEY LLP;ATTN: Patent Group, Suite 1100, 777 - 6th Street, NW, WASHINGTON, DC, 20001, US

(21) Appl. No.: 12/506,826

(22) Filed: Jul. 21, 2009

(51) Int. Cl. A61B 5/08; G09B 23/28

(52) U.S. Cl. 600/529; 434/262

(57) ABSTRACT

A system and method for obtaining measurements of a person's pulmonary diagnostic is described. One embodiment includes a method of providing audible signals related to measurement of pulmonary function, the method comprising receiving a pulmonary input through a receiver, transferring the pulmonary input to a sensor, generating an input signal, using the sensor, based on the pulmonary input, and generating a responsive audible signal through a speaker, wherein the responsive audible signal is based on the input signal. The pulmonary input may comprise, for example, an inhalation, exhalation, gas content reading, or other pulmonary input. The responsive audible signal may comprise a verbal audible signal, a tone, or some combination of both. The responsive audible signal could be for instruction and/or encouragement. The method could further include generating audible instruction signals that are not necessarily related to the pulmonary input.

### FIELD OF THE INVENTION

The present invention relates to systems and methods to assist in obtaining measurements related to the evaluation of a person's pulmonary function.

### BACKGROUND OF THE INVENTION

Pulmonary evaluation equipment allows the measurement of one or more aspects of a person's pulmonary function. Such measurements can be helpful, for example, in determining the extent of a person's healing after a lung injury, in evaluating an asthma patient's lung capacity, or for ensuring pulmonary health during routine physical checkups.

Depending on which characteristic of pulmonary function is being measured, a person may need to exert some effort to provide the necessary input to the equipment being used. For example, a person may need to perform certain maneuvers at certain times during a test. In a hospital, doctor's office, or laboratory, technicians are available to provide instructions and encouragement to the person throughout the duration of the test; however, when using pulmonary evaluation equipment alone or without a trained technician, persons may be unable to complete a test properly, or may be unsure of whether the test results are improving or satisfactory. Moreover, quality of tests may vary in the same pulmonary laboratory or doctor's office due to difference in test technician coaching skills. The same patient tested by different technician in follow-on visits may have inconsistent test results due to variations in coaching techniques.

Some pulmonary evaluation equipment includes a video monitor that can provide visual encouragement. However, such visual feedback does not always result in clear communication with the person, and not all pulmonary evaluation equipment includes a display monitor for visual encouragement.

Accordingly, a system and method are needed to provide users of pulmonary evaluation equipment with non-visual instructions, encouragement, and feedback.

### SUMMARY OF THE INVENTION

Exemplary embodiments of the present invention that are shown in the drawings are summarized below. These and other embodiments are more fully described in the Detailed Description section. It is to be understood, however, that there is no intention to limit the invention to the forms described in this Summary of the Invention or in the Detailed Description. One skilled in the art can recognize that there are numerous modifications, equivalents and alternative constructions that fall within the spirit and scope of the invention as expressed in the claims.

The present invention relates to pulmonary diagnostic. In one exemplary embodiment, the present invention can include a method of providing audible signals related to measurement of pulmonary function, the method comprising receiving a pulmonary input through a receiver, transferring the pulmonary input to a sensor, generating an input signal, using the sensor, based on the pulmonary input, and generating a responsive audible signal through a speaker, wherein the responsive audible signal is based on the input signal. The pulmonary input may comprise, for example, an inhalation, exhalation, gas content reading, or other pulmonary input. The responsive audible signal may comprise a verbal audible signal, a tone, or some combination of both. The responsive audible signal could be for instruction and/or encouragement. The method could further include generating audible instruction signals that are not necessarily related to the pulmonary input. For example, the audible instruction signals could be based on the type of pulmonary test being performed, and the timing requirements of such a test, and/or the age and health condition of the user.

In another exemplary embodiment, the present invention can include a system for measuring pulmonary function, comprising a receiver configured to accept an input from a pulmonary system of a person, an input carrier, connected to the receiver and configured to carry the input from the receiver to a sensor, the sensor configured to measure a value of the input, a processor connected to the sensor, wherein the processor is configured to select an audible signal in response to the value of the input, and a speaker connected to the processor, wherein the speaker is configured to generate audible feedback based on the audible signal. This system could be incorporated into a plethysmograph or used as a separate handeld device. Other features, such as a display screen, user interface, test gas chamber, shutter, or other feature related to a pulmonary test may be further incorporated into the system. Moreover, features that allow data stored on the system to be transmitted to other locations could be easily incorporated into the system.

As previously stated, the above-described embodiments and implementations are for illustration purposes only. Numerous other embodiments, implementations, and details of the invention are easily recognized by those of skill in the art from the following descriptions and claims.

### BRIEF DESCRIPTION OF THE DRAWINGS

Various objects and advantages and a more complete understanding of the present invention are apparent and more readily appreciated by reference to the following Detailed Description and to the appended claims when taken in conjunction with the accompanying Drawings, wherein:

FIG. 1 shows a system configured to accept an input from a person, analyze the input, and generate audible feedback corresponding to the input;

FIG. 2 shows a system configured to be held in a person's hand;

FIG. 3 shows a method of measuring pulmonary function and generating responsive audible feedback;

FIG. 4 shows one embodiment of the step of accepting an input from a person;

FIG. 5 shows one embodiment of the step of analyzing a person's input;

FIG. 6 shows one embodiment of the step of providing audible feedback to a person in response to the person's input;

FIG. 7 shows an exemplary flow chart for a spirometry test consistent with the present invention;

FIG. 8 shows an exemplary breathing pattern diagram for a spirometry test;

FIG. 9 shows a diagram of an exemplary breathing pattern for body plethysmography; and

FIG. 10 shows an exemplary breathing diagram for single-breath DLCO.

### DETAILED DESCRIPTION

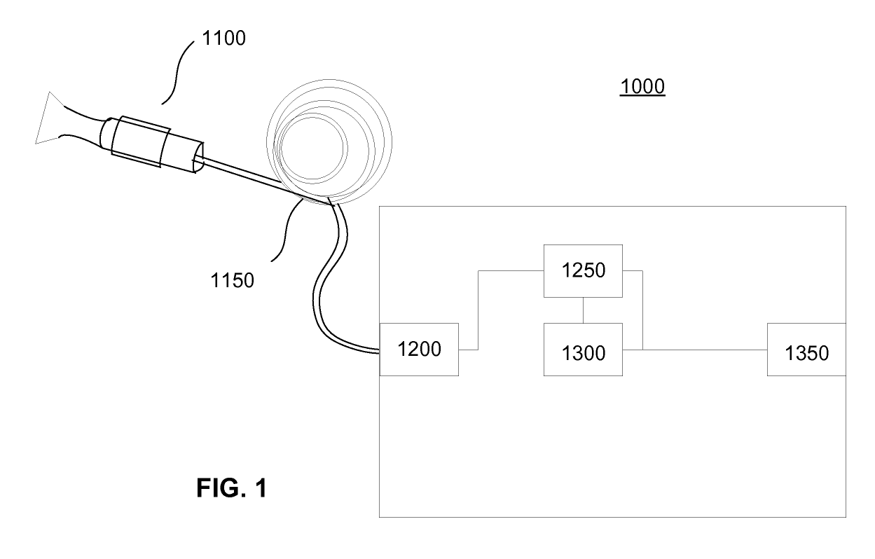

Referring now to the drawings, where like or similar elements are designated with identical reference numerals throughout the several views, FIG. 1 shows one embodiment of a pulmonary measurement, or pulmonary evaluation, machine 1000 in accordance with the present invention. The pulmonary measurement machine 1000 includes a receiver 1100, such as a mouthpiece, connected to an input carrier 1150, which in turn is connected to a sensor 1200. The sensor 1200 is connected to a processor 1250, and the processor 1250 is connected to an audible signal storage unit 1300. The processor 1250 is also connected to an audio speaker 1350.

Various embodiments of each of these elements will be recognized by persons of skill in the art. For example, the receiver 1100 may comprise a mouthpiece, a tube, a plurality of tubes, a receptacle, a sensor, or another appropriate device for receiving a pulmonary input (meaning an input from a person's pulmonary system) from a person using a pulmonary measurement machine 1000. A receiver 1100 may be designed to accept as a pulmonary input an inhalation or exhalation from a person, or it may be designed to be inserted into a person's lungs, or near a person's diaphragm, or anywhere else where a pulmonary input may be received. A person may or may not have to exert physical activity to effect or create the pulmonary input.

Various embodiments of the input carrier 1150 are possible as well. It may be, for example, a tube, or it may be a receptacle configured to removably attach first to the receiver 1100, where it can collect a pulmonary input, and then to pulmonary measurement machine 1000 in such a way as to expose the sensor 1200 to the pulmonary input. In some embodiments, a receiver 1100 and input carrier 1150 may be unnecessary; for example, the sensor 1200 may be configured to be placed inside a person's lungs, and may be capable of transmitting a voltage through a wire to the processor 1250.

As with the other elements of the pulmonary measurement machine 1000, various embodiments of the sensor 1200 are also possible. The sensor 1200 may be a pressure sensor, flow sensor, viscosity sensor, density sensor, humidity sensor, mechanical sensor, chemical sensor, pneumotach, hot-wire anemometer, or any of a variety of other sensors known to persons of skill in the art. The processor 1250 may be any processor capable of accepting an input from a sensor 1250, calculating a value in one or multiple steps, and, alone or in combination with other hardware, choosing a responsive audible signal (meaning an audible signal that corresponds in some way to the pulmonary input itself or to the value calculated by the processor 1250) from an audible signal storage device 1300 and causing the responsive audible signal to be played on a speaker 1350.

Referring still to FIG. 1, the audible signal storage device 1300 may be a hard drive, a flash drive, or a pre-programmed read-only memory module. It may also be a media drive capable of accepting and reading removable storage devices, such as removable flash drives, DVDs, CDs, or diskettes; indeed, the audible signal storage device 1300 may not have any permanent storage at all, but rather may contain stored audible signals solely on removable storage devices. In addition, the speaker 1350 may be fixedly attached to the pulmonary measurement machine 1000, or it may be removably attached to the pulmonary measurement machine 1000. The speaker 1350 may, for example, comprise removable headphones, or the speaker 1350 may be built in to the pulmonary measurement machine 1000. Many other variations of these and other components of the pulmonary measurement machine 1000 are possible and will be understood by those skilled in the art.

In one embodiment of the pulmonary measurement machine 1000 shown in FIG. 1, a user of the pulmonary measurement machine 1000 must provide a pulmonary input, such as an exhalation. For example, the user may place his mouth over, and exhale into, a receiver 1100 embodied as a mouthpiece. The user's exhalation is then carried to the sensor 1200 via the input carrier 1150. Consistent with the present invention, the sensor 1200 can be selected and/or configured to produce a voltage that varies in magnitude depending on the characteristic to be measured. For example, in a first embodiment, the sensor 1200 could be an enclosed propeller anemometer that produces a low voltage when the air passing through the anemometer causes a low rotational velocity, and produces a high voltage when the air passing through the anemometer causes a high rotational velocity. In another embodiment, the sensor 1200 could be a pitot tube that produces a low voltage when the pressure differential between the static and dynamic pressures is low, and produces a high voltage when the pressure differential is high. A variety of sensors are known to persons of skill in the art and can be used to aid in determining the value of a variety of pulmonary characteristics.

Characteristics of a pulmonary input that may be measured using a system such as the pulmonary measurement machine 1000 include spirometric flow, spirometric volume, and oxygen concentration, among other things.

The processor 1250 can use the signal produced by the sensor 1200 in response to the pulmonary input to calculate a desired value. For example, if the desired value is the total exhalation volume and the sensor is an enclosed propeller anemometer, the processor 1250 can use the voltage to determine the rotational velocity of the propeller, which it can use to calculate the air speed at each moment during the exhalation. By integrating the air speed over time and multiplying the result by the cross-sectional area of the anemometer, the total volume can be closely approximated. Other values of characteristics can be calculated using appropriate sensors and formulas. Once the processor 1250 has calculated the desired value or values, it can locate within the audible signal storage unit 1300 one or more responsive audible signals to play through the speaker 1350 in response to the calculated value or values. The processor may also locate, within the audible signal storage unit 1300, one or more audible instruction signals to play through the speaker 1350 in response to factors other than pulmonary inputs. For example, the processor 1250 could run a program where some audible instruction signals are selected based on factors such as time, age, type of pulmonary test being performed and/or other non-pulmonary input variables. Responsive audible signals may also be selected on a combination of both a pulmonary input and some other non-pulmonary input factor (such as time, heart rate, age of user, etc.). Moreover, the calculated value used to select the responsive audible signal(s) may directly relate to a characteristic of the pulmonary input, or it may indirectly relate to a characteristic of the pulmonary input. For example, the calculated value could be a total volume, or it could be a rate of change of total volume, or it could be a difference between two or more measurements of total volume.

The responsive audible signal chosen by the processor 1250 may, for example, be a tone that increases in pitch over time in proportion to the magnitude of the value representing the total volume of air exhaled by the user, such that as the user exhales the tone generated by the speaker 1350 rises in pitch. The user can know whether his total volume is increasing from use to use by comparing the highest pitches achieved in each successive use of the pulmonary measurement machine 1000. Similarly, an audible instruction signal chosen by the processor 1250 may, for example, be a tone that increases in pitch over time in proportion to the magnitude of the value representing the volume of air the user should be exhaling. In this way, the audible instruction signal could provide instructions as to how the user is to perform a given test. For more complicated pulmonary test types, such as an Inspiratory Capacity test for a Chronic Obstructive Pulmonary Disease patient, or simpler tests, such as a Peak Expiratory Flow (PEF) test or Forced Expiratory Volume in 1 second (FEV1) test, the audible signal storage unit 1300 may need to provide verbal instructions or verbal instructions in combination with tones and other audio cues. As will be understood by those skilled in the art, some audible signals would actually start before any pulmonary inputs are received, while others will be used during and after a pulmonary input or pulmonary inputs.

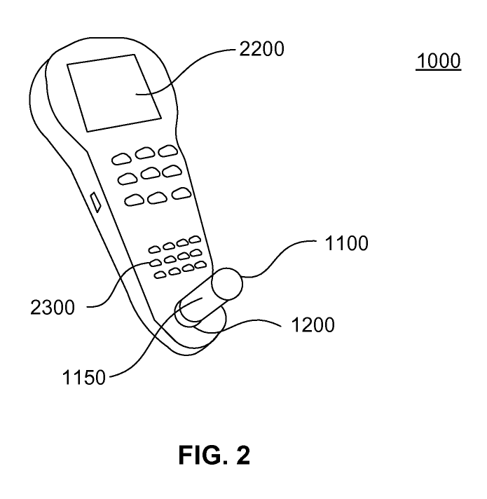

Referring now to FIG. 2, in another embodiment of the invention a handheld pulmonary measurement machine 1000 can include a receiver 1100, an input carrier 1150, a sensor 1200, a processor 1250 (internal not shown), an audible signal storage unit 1300 (internal not shown), and a speaker 1350 (not shown). Also shown in FIG. 2 is a display 2200 and interface 2300. A person using this handheld pulmonary measurement system 2000 may, among other things, exhale into the receiver 1100 to obtain a peak flow rate measurement. The exhalation can then be carried by the input carrier 1150 to the sensor 1200. The sensor 1200 generates a signal, such as a voltage, current, or magnetic field strength, based on a characteristic of the input; for a peak flow rate measurement, the signal would correspond to the speed of the air passing over or through the sensor. The processor 1250 can then use the voltage to calculate the actual flow rate. The processor 1250 can compare the present calculated value with the highest previously calculated value to determine whether the flow rate has peaked. Once the input has terminated and a peak flow rate has been established, the processor 1250 can choose, from the audible signal storage unit 1300, a responsive audible signal that corresponds to the result. At the processor's 1250 command, the chosen audible signal can be sent to the speaker 1350 for playback. The responsive audible signal chosen in this situation may be a verbal signal that communicates the results of the comparison, such as “Congratulations! Your peak flow rate is [insert value]!” if the peak flow rate is favorable or “Your peak flow rate is in the yellow zone” if the peak flow rate is lower than desired. The pulmonary measurement machine 1000 could also store values for successive uses, allowing a user or a doctor to review measurements over time.

Here as with other embodiments, a variety of audible signals (whether responsive audible signals or audible instruction signals) may be used consistent with the present invention, and those signals may be chosen for a variety of reasons. An audible (tonal or verbal) signal may be provided to patients to improve accuracy, consistency and repeatability of pulmonary function tests by encouraging patients to achieve maximal effort, guiding patients through complex maneuvers, reducing patient anxiety and reducing dependency on coaching skills of test technicians. For example, the responsive audible signal generated as audible feedback to the user may be a tone, a musical composition, verbal encouragement, verbal instructions, or some other sound. The responsive audible signal may be played before the input, during the input or after the input is completed. The audible signal may change, either continuously over time or in response to various values, including measured pulmonary inputs, heart rate, or time. The audible signal could be generated according to a pre-determined timing sequence, triggered by occurrence of specific events, or created in response to patient's input during test. For example, a measurement of the amplitude of spirometric flow may trigger audible feedback in the form of a tone. Or, if an inhalation or exhalation that lasts a specific amount of time is needed, a monotone could play for the duration of that time period, or the tone could increase in volume or pitch as time passes until it stops when the necessary time period has ended. Also, the audible signal storage unit can contain instructions for producing an audible signal in addition to or instead of only storing previously produced audible signals. For example, in addition to containing a stored congratulatory message, the audible signal storage unit can contain instructions for producing a tone that varies in pitch according to the calculated value of the input.

The particular responsive audible signal played in response to a value or values calculated from a pulmonary input can be chosen for a variety of reasons, including to encourage the user to adjust the input, to provide instructions, to provide information about the input, or for other reasons known to persons of ordinary skill in the art. For example, the responsive audible signal could be motivational feedback in the form of verbal encouragement. If a favorable rate of change occurs, the user receives congratulatory audible feedback (i.e. “Good job!” or “Keep up the good work!”), and if an unfavorable rate of change occurs, the user receives encouraging audible feedback (i.e. “Try a little harder!” or “You can do better! Let's try again!”). Or, the audible instruction signal might comprise instructions relevant to the pulmonary test being administered (i.e. “inhale now” or “continue exhaling” or “breathe deeply”).

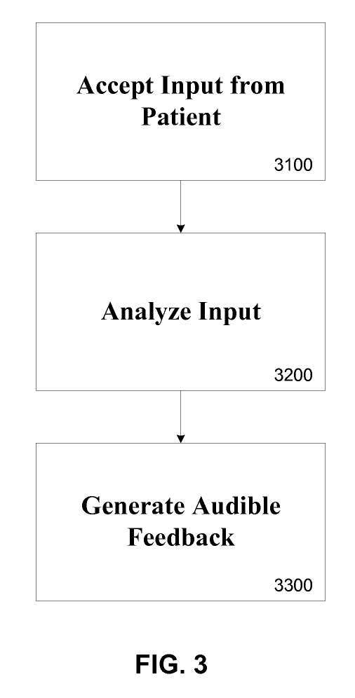

Referring now to FIG. 3, a method for generating audible feedback while measuring pulmonary function comprises the steps of accepting a pulmonary input from a user 3100; analyzing the pulmonary input to measure a value thereof 3200, and generating audible feedback to the user in response to the measured value 3300. This embodiment is exemplary only, and persons of ordinary skill in the art will recognize other ways of accomplishing the present invention.

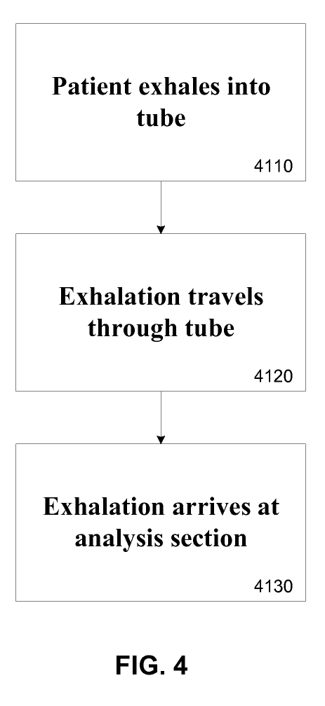

Referring now to FIG. 4, in one embodiment, accepting an input from a user 3100 may comprise the steps of a user exhaling into a mask 4110, the exhalation being carried through a tube away from the mask and towards a sensor 4120, and the exhalation arriving at the sensor 4130. In other embodiments, the sensor may receive the input directly. For example, if the oxygen concentration within a user's lungs is to be measured, a sensor may be inserted directly into the user's pulmonary system, with wires extending from the sensor to a processing unit. In another embodiment, a container may be used to capture an input from a user, such as an air sample. The container could then be removably attached to a pulmonary evaluation unit in such a way that the contents of the container would come into contact with a sensor. Persons of ordinary skill in the art will recognize other ways of accomplishing the step of accepting an input from a user 3100.

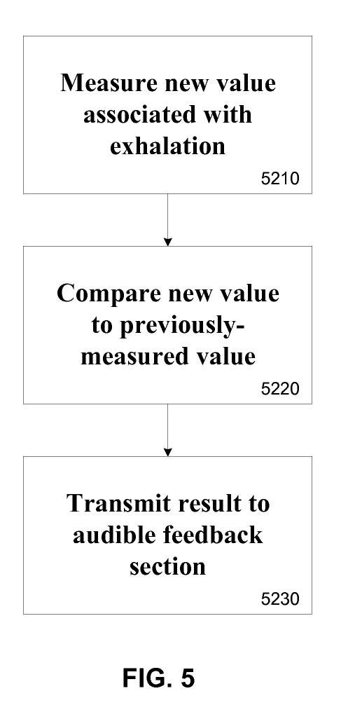

Referring now to FIG. 5, the step of analyzing the input 3200 may comprise the steps of using a sensor to generate a voltage that reflects a characteristic of an exhalation 5210, measuring the voltage with the processor 5220, calculating a value of the characteristic of the exhalation using the measured voltage 5230, and repeating these steps throughout the duration of the input. Many other embodiments are also possible. For example, the steps of using a sensor to generate a voltage that reflects a particular characteristic of an exhalation 5210, measuring the voltage with the processor 5220, and calculating a value of the characteristic of the exhalation using the measured voltage 5230 may not occur until after the input has been completed. Or, these steps may occur only once during the input. In yet another embodiment, the step of analyzing the input 3200 may further include the step of comparing the calculated value with a pre-set or stored value, or the step of comparing the present calculated value with a previously calculated value. Still another embodiment may include the steps of using multiple sensors to generate multiple voltages that reflect multiple physical characteristics of an exhalation, measuring the multiple voltages with one or more processors, and calculating the value of the particular physical characteristic associated with each voltage. This embodiment may further comprise the step of calculating additional values that in some way quantify or describe the user's pulmonary evaluation system yet cannot be directly measured or directly calculated from a sensor's input.

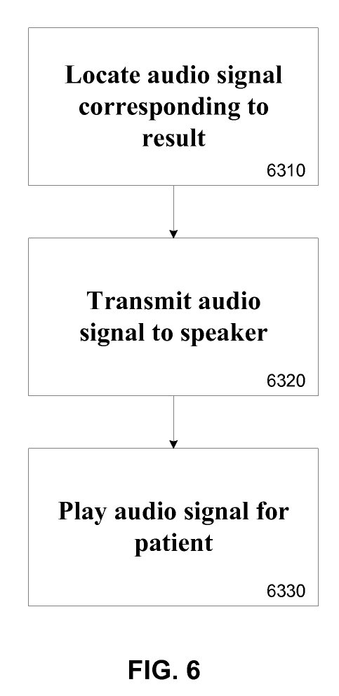

Referring now to FIG. 6, the step of generating audible feedback in response to the pulmonary input 3300 may further comprise the steps of the processor choosing a responsive audible signal that corresponds, at least in part, to the measured value 6310, transmitting the responsive audible signal to the speaker 6320, and playing the responsive audible signal for the user 6330. Again, many embodiments of the step of generating audible feedback in response to the user's input 3300 will be recognized by persons of ordinary skill in the art. For example, the processor may choose multiple audible signals to play in response to the input, may choose to vary a single audible signal as successive values are calculated throughout the duration of the input, or may choose an audible signal to play for each of multiple calculated values.

The above aspects, and other aspects not specifically described, can be modified in a variety of ways, only some of which are described herein. One modification or addition that may be desirable is the ability to take multiple measurements over the duration of the user's input, such that a processor 1250, for example, can choose a new audible signal, or can change a previously-chosen audible signal, based on values updated either continuously (i.e. in real-time) or at a set interval (i.e. every seven seconds). In this way the audible feedback generated to the user can vary in response to changing values (such as time, pulmonary input values, heart rate, etc.). For example, for pulmonary input value, repeated measurement also allows for a determination of whether the pulmonary input characteristic being measured is increasing or decreasing, and appropriate responsive audible feedback can be generated accordingly. Since it may take more time to play a given responsive audible signal than it takes to make multiple measurements, a set number of successive values can be averaged. This allows each responsive audible signal to be chosen based on an average value calculated at a slow enough frequency to ensure that each responsive audible signal has sufficient time to be played before playback of a new responsive audible signal is initiated.

Pulmonary evaluations coupled with audio feedback need not be devoid of other incentive devices; for example, the evaluations could include visual feedback as well. Further, the evaluations may be conducted using a stationary device, a portable device, or even a handheld device.

One application of the present invention is for use in spirometry. Spirometry is a test that measures how an individual inhales or exhales volumes of air as a function of time. For example, spirometry can be used to measure the changes in lung volumes (but not the absolute lung volumes) as well as flow rates achieved by an individual during inspiration and expiration. This is useful for detecting, characterizing, and quantifying the severity of lung diseases. However, while spirometry can be undertaken using many different types of equipment, it requires cooperation between the subject and whomever is conducting the test, and the results obtained will depend on technical as well as personal factors. (Miller M R, Hankinson J, Brusasco V, et al. Standardisation of Spirometry, Eur Respir J 2005; 26: 319-338.)

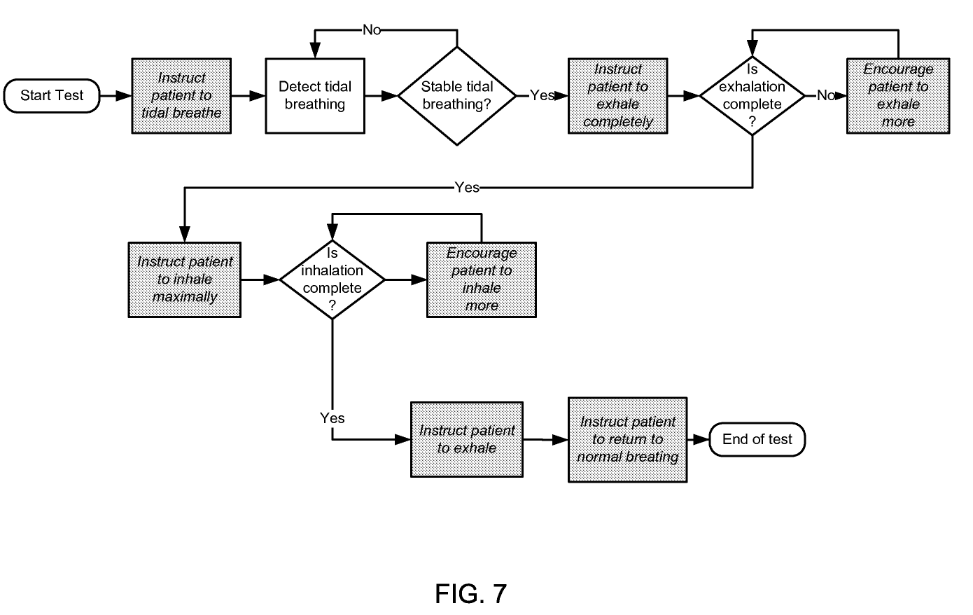

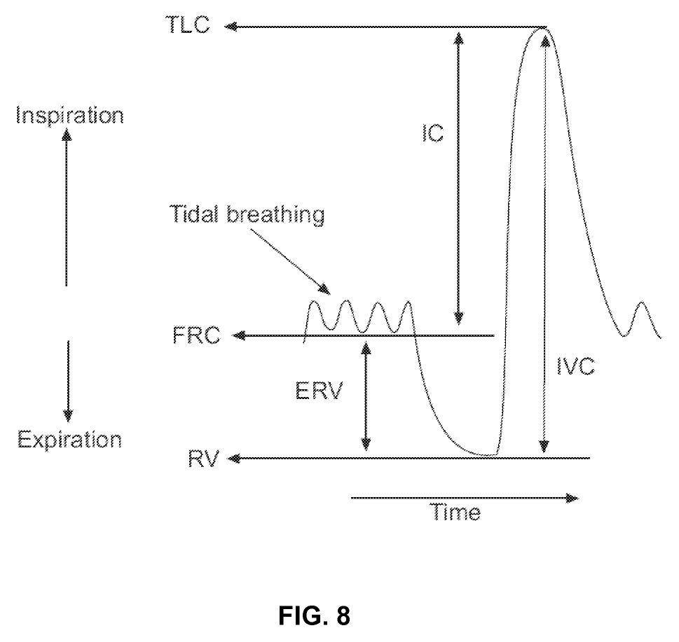

Typical spirometry measurements will require patient to: (a) breath normally (tidal breathing) initially, and (b) perform maximal inhalation and/or followed by maximal exhalation (or vice versa) after a stable tidal breathing pattern is achieved. During maximal inhalation or exhalation, test technician would provide various verbal instructions to a patient such as inhale rapidly, inhale more, exhale rapidly, keep on blowing, etc. FIG. 7 provides an exemplary flow chart for spirometry consistent with the present invention and FIG. 8 shows an exemplary breathing pattern diagram for spirometry. (Miller M R, Hankinson J, Brusasco V, et al. Standardisation of spirometry. Eur Respir J 2005; 26: 319-338, FIG. 11.) In FIG. 7 shaded boxes indicate audible signals provided to users. In one embodiment, a user could select a spirometry test (for example, the user could select a spirometry test option, from multiple pulmonary test options presented on the display 2200, using the user interface 2300) from a selection of different types of spirometry test options on the pulmonary measurement machine 1000. Based on the spirometry test selected, not shown in FIG. 7, audible instructions could be provided to remind the use of correct posture, and the proper efforts to achieve best results. This can be provided in conjunction with or independent of pictorial instructions. As the test starts, the processor 1250 could select audible instruction signals that coach the user (patient) to perform tidal breathing. As the user (patient) begins to breathe into the receiver 1100 the pulmonary measurement machine 1000 will detect the pulmonary input of the user and determine if the tidal breathing is stable. Further checks, and instruction, can be repeated until stable tidal breathing is detected. Various parameters can be set to determine if stable tidal breathing has been achieved. For example, in one embodiment, the pulmonary measurement machine 1000 could detect at least four (4) tidal breaths where the end-tidal volumes of the four (4) successive breaths are within a predetermined value (e.g., 50 mL). Those of skill in the art will readily understand other ways to determine whether tidal breathing has been achieved.

After tidal breathing is established, the pulmonary measurement machine 1000 can select and provide audible instruction signals for complete exhalation and inhalation. As the patient is exhaling or inhaling, encouragement can be provided in various forms, such as “Good job, keep going.” Alternately, an acoustic tone with increasing frequency will “incentivize” patient to provide more effort. This encouragement and instruction can continue until a minimum inhalation or exhalation rate is achieved. For example, once the pulmonary measurement machine 1000 detects less than 0.025 L of air is expired/inhaled in 1 second or patient has tried to exhale for more than 6 seconds (3 seconds for patients under 10 years old), the program can move to the next step until testing is complete.

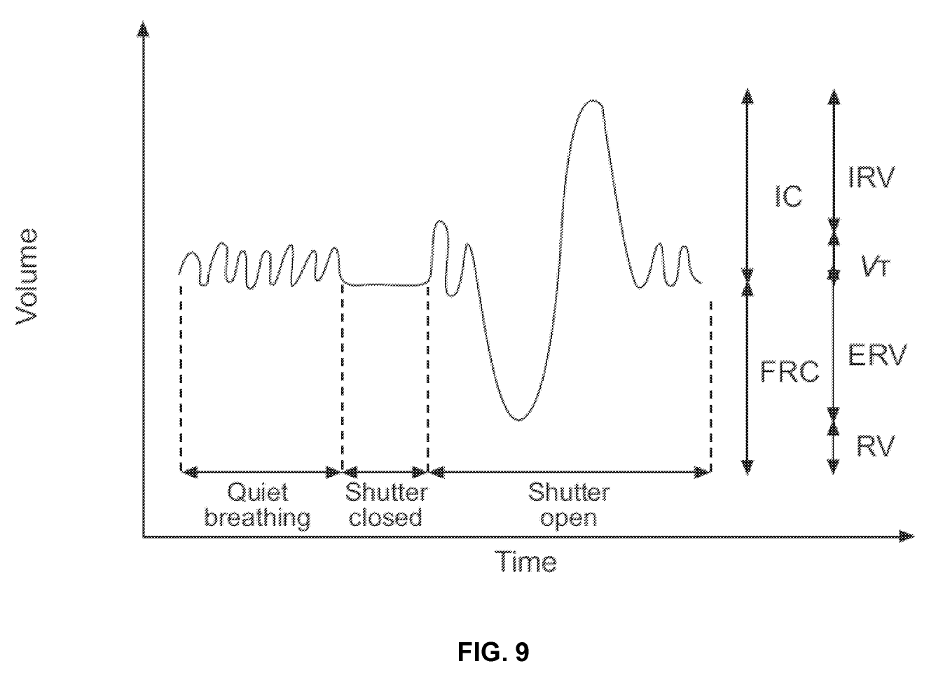

In another embodiment, the present invention could be used to instruct and coach a user through a series of maneuvers required for body plethysmography. FIG. 9 shows a diagram of an exemplary breathing pattern for body plethysmography. (Wanger J, Clausen J L, Coates A, et al. Standardisation of the measurement of lung volumes. Eur Respir J 2005; 26: 511-522.) As shown, if a body plethysmography test is selected, the user would be instructed to perform normal (quiet) breathing into the pulmonary measurement machine 1000. It will be understood by those skilled in the art that for body plethysmography, the pulmonary measurement machine 1000 will be incorporated into a plethysmograph. Once normal breathing is established, the processor could select the audible instruction signals for panting. For example, in one embodiment, a metronome like tone could be used to guide user to pant at the appropriate rate (e.g., between 0.5 to 1.5 Hz or below 1.0 Hz). For some tests, the pulmonary measurement machine 1000 could also measure the flow rate during panting and provide a responsive audible signal to pant shallower if flow is greater than 0.5 L/s. For FIG. 9, it shows that the user will pant against a closed shutter. For this embodiment, the pulmonary measurement machine 1000 could comprise a shutter that can be opened and closed in order to allow for various types of testing. The shutter could be opened or closed automatically, manually, or both. Such a shutter could be incorporated into other embodiments where a shutter is required. Maximal exhalation and maximal inhalation will also benefit from approaches similar to those for spirometry.

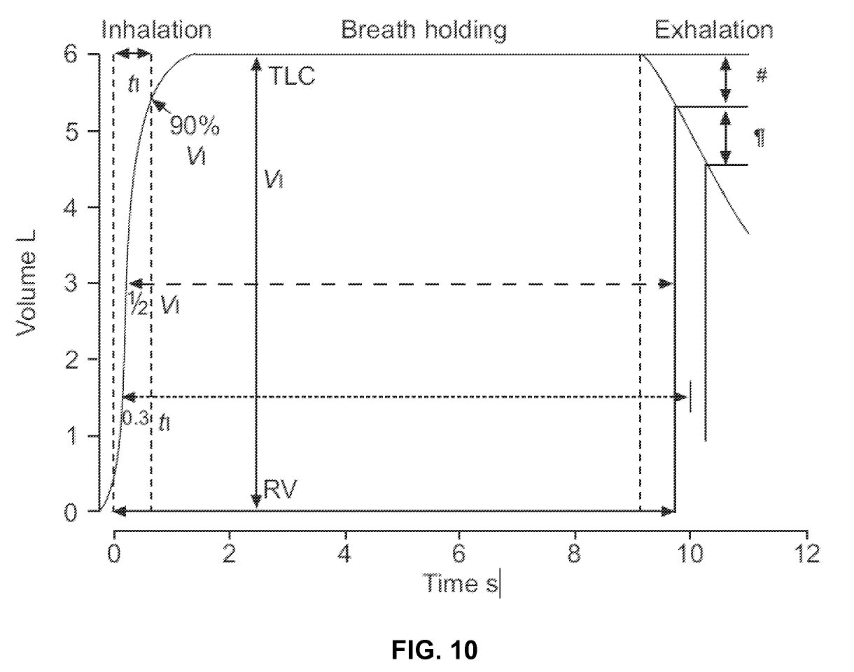

In another embodiment, the present invention could be used to assist with measuring diffusing capacity. Diffusing capacity is a measure of lung's capacity to exchange gas across the alveolar-capillary interface. The standard approach is to measure the rate of carbon monoxide uptake when a small amount of CO is included in the breathing gas. One of the most common CO uptake method is the single breath CO diffusing capacity measurement. FIG. 10 represents an exemplary breathing diagram for single-breath DLCO. (MacIntyre N, Crapo R O, Viegi G, et al. Standardisation of the single-breath determination of carbon monoxide uptake in the lung. Eur Respir J 2005; 26: 720-735.) For the present invention, a single breath DLCO program could be initiated by a user and the pulmonary measurement machine 1000 could provide initial instructions based on the program. After any introductory audibly instruction signals, the user could be instructed to begin tidal breathing. Tidal breathing should be carried out for a sufficient time to assure that the subject is comfortable with the receiver 1100. Once sufficient tidal breathing has been established, the processor 1250 could begin instructions for unforced exhalation to residual volume (RV). In some embodiments, the pulmonary measurement machine 1000 could be programmed with information about the user in order to control testing. In some embodiments, the user could be prompted to enter certain information before testing begins. For example, here the exhalation may be limited to 6 seconds for users with obstructive diseases. Those of skill in the art will readily understand how other user information could be used to select the proper audible instruction signals and responsive audible signals.

Once exhalation is complete, the user could be provided with instructions to inhale rapidly and maximally. For this embodiment, the pulmonary measurement machine 1000 could further comprise a test gas chamber (not shown) which can release a specified amount of inhale test gas through the receiver 1100 so it is inhaled by the user. For single-breath DLCO for example, the inhale test gas would comprise typically around 3000 ppm of CO. Once the inhalation had dropped below a certain rate, or lasted a certain period of time, the processor 1250 selects audible instruction signal(s) that encourage patient to hold breath for 10 seconds (+/−2 seconds). For example, this could include multiple forms of encouragement at the same time as, or independent from, a countdown. At the end of the breath hold, audible instruction signals would be provided to exhale rapidly. For the exhalation, the pulmonary measurement machine 1000 could further comprise a gas analyzer (single-sample or continuous) to determine the amount of CO present in the exhalation. This gas analyzer could be incorporated as part of the sensor 1200 or could be designed separately into the machine 1000.

In conclusion, the present invention provides, among other things, a system and method for providing audible feedback to a user of a pulmonary evaluation machine. Those skilled in the art can readily recognize that numerous variations and substitutions may be made in the invention, its use and its configuration to achieve substantially the same results as achieved by the embodiments described herein. Accordingly, there is no intention to limit the invention to the disclosed exemplary forms. Many variations, modifications, and alternative constructions fall within the scope and spirit of the disclosed invention as expressed in the claims.

What is claimed is:

1. A method of providing audible signals related to measurement of pulmonary function, the method comprising:
receiving a pulmonary input through a receiver;
transferring the pulmonary input to a sensor;
generating an input signal, using the sensor, based on the pulmonary input; and
generating a responsive audible signal through a speaker, wherein the responsive audible signal is based on the input signal.

2. The method of claim 1, wherein the pulmonary input comprises an exhalation.

3. The method of claim 1, wherein the responsive audible signal comprises a verbal responsive audible signal.

4. The method of claim 1, wherein generating the responsive audible signal through the speaker comprises:
determining a value of a characteristic of the pulmonary input using a processor, wherein the processor determines the value of the characteristic of the pulmonary input based on the input signal; and
generating the responsive audible signal through the speaker based on the value of the characteristic of the pulmonary input.

5. The method of claim 4, wherein determining the value of the characteristic of the pulmonary input using the processor comprises:
determining a flow rate of the pulmonary input using the processor, wherein the flow rate is based on the input signal.

6. The method of claim 1, wherein generating the responsive audible signal through the speaker comprises:
determining a first input level using the input signal at a first point in time;
determining a second input level using the input signal at a second point in time; and
generating the responsive audible signal through the speaker based on the difference between the first input level and the second input level.

7. The method of claim 1, wherein the responsive audible signal comprises a motivational responsive audible signal.

8. The method of claim 1, further comprising:
generating an audible instruction signal through the speaker.

9. The method of claim 1, further comprising:
receiving a pulmonary test type selection; and
generating an audible instruction signal through the speaker based on the pulmonary test type selection.

10. A method of providing audible feedback while measuring pulmonary function, the method comprising:
accepting an input from a pulmonary system of a person;
analyzing the input, within a device, to measure a value of the input; and
generating, using the device, an audible feedback to the person in response to the value of the input.

11. The method of claim 10, wherein analyzing the input to measure the value of the input comprises:
analyzing the input within the device to measure the value of the input two or more times in succession.

12. The method of claim 11, wherein generating the audible feedback to the person in response to the value of the input comprises:
generating, using the device, the audible feedback to the person in response to each of the measured values of the input.

13. The method of claim 10, wherein the input comprises an inhalation.

14. The method of claim 10, further comprising:
generating an audible instruction to the person.

15. The method of claim 14 wherein generating an audible instruction to the person comprises:
receiving a pulmonary test selection from the person; and
selecting the audible instruction based on the pulmonary test selection.

16. The method of claim 15, further comprising:
receiving identifying information from the person; and
selecting the audible instruction based on the pulmonary test selection and the identifying information.

17. The method of claim 16 wherein the identifying information is selected from a group consisting of an age and a health condition.

18. The method of claim 10 wherein generating, using the device, the audible feedback to the person comprises:
receiving a pulmonary test selection from the person; and
generating, using the device, an audible feedback instruction to the person based on the pulmonary test selection and the value of the input.

19. A system for measuring pulmonary function, comprising:
a receiver, configured to accept an input from a pulmonary system of a person;
an input carrier, connected to the receiver and configured to carry the input from the receiver to a sensor, the sensor configured to measure a value of the input;
a processor connected to the sensor, wherein the processor is configured to select an audible signal in response to the value of the input; and
a speaker connected to the processor, wherein the speaker is configured to generate audible feedback based on the audible signal.

20. The system of claim 19, wherein the system is contained in a handheld device.
